(19) United States
(12) Patent Application Publication
(10) Pub. No.: US 2011/0305652 A1
(43) Pub. Date: Dec. 15, 2011

(54) Anionic Surfactant Stable Cocoamide Monoethanolamine (CMEA) Composition

(76) Inventors: Jennifer Elaine HILVERT, Cincinnati, OH (US); Marco Caggioni, Cincinnati, OH (US); Benjamin Scott Schlagheck, Cincinnati, OH (US); Douglas Allan Royce, Sunman, IN (US)

(21) Appl. No.: 13/156,790

(22) Filed: Jun. 9, 2011

Related U.S. Application Data
(60) Provisional application No. 61/352,997, filed on Jun. 9, 2010.

(51) Int. Cl. A61K 8/92; A61K 47/20; A61Q 5/00; A61Q 17/04; A61P 17/00; A61Q 19/00; A61K 47/16; A61K 47/24

(52) U.S. Cl. 424/59; 514/784; 514/788; 424/70.1; 424/70.22; 424/70.24; 424/70.23; 424/70.19; 514/18.8

(57) ABSTRACT

Disclosed herein is a composition containing liquid high activity micellar or lamellar phase liquid crystals of cocamide monoethanolamine (CMEA) and a detersive surfactant. The composition of the invention can be added to any personal care composition to form a personal care product and enable CMEA blending at room temperature. Also disclosed herein are methods for making the high activity CMEA micellar or lamellar phase liquid crystal composition.

### CROSS REFERENCE TO RELATED APPLICATION

This application claims the benefit of U.S. Provisional Application No. 61/352,997 filed Jun. 9, 2010.

### FIELD OF THE INVENTION

One embodiment of the present invention relates to a composition of high activity liquid form of cocamide monoethanolamine (CMEA) enabling room temperature blending of CMEA, methods of making these novel compositions, and use of the CMEA liquid compositions to prepare personal care products.

### BACKGROUND OF THE INVENTION

Cocamide monoethanolamine (CMEA) is a waxy solid, nonionic, co-surfactant often used as a foam booster, viscosity builder, and/or emulsifying agent in personal care products. When CMEA is incorporated into a personal care composition, it solubilizes to form micelles by co-assembling with detersive surfactants that are present in the composition. This solubilization of CMEA requires heating, which limits the process conditions that can be used to form personal care products. For example, in some traditional processes, the solubilization of CMEA into a shampoo composition requires the preparation of a hot slurry of CMEA that is added to a hot shampoo composition.

Suppliers of CMEA have attempted to address the problem of having to heat every personal care composition to which CMEA is added by providing pre-solubilized compositions of CMEA. However, these compositions are not only expensive, but they all contain high levels of amphoteric surfactants (e.g., cocoamidopropyl betaine, sodium cocamphopropionate), which severely narrow subsequent formulation options.

### SUMMARY OF THE INVENTION

Described herein are compositions composed of micellar or lamellar phase liquid crystals of cocamide monoethanolamine (CMEA), a detersive surfactant, and water. As the composition is composed of micellar or lamellar phase liquid crystals is substantially free of hexagonal phase liquid crystals on the dissolution path to final personal care product composition. In some embodiments, the composition of the invention is phase stable at a temperature range of about 20° C. to about 30° C. In one embodiment the composition has a molar ratio of CMEA to detersive surfactant of about 1:1 to about 1:20, in another embodiment from about 1:1 to about 1:5, and in one embodiment a molar ratio of CMEA to water of about 1:50 to about 1:1000, in another embodiment from about 1:50 to about 1:250. In some embodiments, the composition of the invention is composed of about 5 wt. % to about 25 wt % of CMEA, about 10 wt. % to about 30 wt. % of detersive surfactant, and about 55 wt. % to about 85 wt. % water, based on the total weight of the composition.

Another aspect of the invention is a method for making compositions composed of cocamide monoethanolamine (CMEA), a detersive surfactant, and water. In this method, CMEA is added to a solution of a neutralized, preserved detersive surfactant at a temperature of about 50° C. to about 86° C. to form a composition composed of lamellar phase liquid crystals. This composition is then optionally cooled. In some embodiments, the composition is cooled to about 27° C. to about 40° C.

Another aspect of the invention is a method for making a personal care product. In this method, a personal care composition that contains a component selected from the group consisting of conditioning agents, natural cationic deposition polymers, synthetic cationic deposition polymers, anti-dandruff agents, gel networks, particles, suspending agents, paraffinic hydrocarbons, propellants, viscosity modifiers, dyes, non-volatile solvents, water soluble diluents, water insoluble diluents, pearlescent aids, foam boosters, surfactants, pediculocides, pH adjusting agents, perfumes, preservatives, chelants, proteins, skin active agents, sunscreens, UV absorbers, vitamins, and mixtures thereof, is added to a composition composed lamellar phase liquid crystals of cocamide monoethanolamine (CMEA), a detersive surfactant, and water.

### BRIEF DESCRIPTION OF THE DRAWINGS

While the specification concludes with claims particularly pointing out and distinctly claiming the subject matter that is regarded as the present invention, it is believed that the invention will be more fully understood from the following description taken in conjunction with the accompanying drawings. Some of the figures may have been simplified by the omission of selected elements for the purpose of more clearly showing other elements. Such omissions of elements in some figures are not necessarily indicative of the presence or absence of particular elements in any of the exemplary embodiments, except as may be explicitly delineated in the corresponding written description. None of the drawings are necessarily to scale.

FIGS. 1a-c compare the traditional method (FIG. 1a) for solubilizing cocamide monoethanolamine (CMEA) into personal care compositions that have a detersive surfactant (e.g., sodium lauryl sulfate (SLS)), with the method according to an embodiment of the invention (FIG. 1b), which involves the preparation of an intermediate composition containing high activity liquid form of CMEA and a detersive surfactant, and its subsequent dilution into a personal care composition. The ternary phase diagram shown in FIG. 1c illustrates the relationship between the concentrations of CMEA, detersive surfactant (e.g., SLS), and water at different points in the traditional method for incorporating CMEA into a personal care composition, and in the method of the invention. Point 1 depicts 100 wt. % CMEA. Point 2 depicts the concentrations of CMEA, detersive surfactant (e.g., SLS), and water that result in a lamellar liquid crystal phase. Point 3 depicts the concentrations of CMEA, detersive surfactant (e.g., SLS), and water that result in micelles.

FIG. 2 is a ternary phase diagram of cocamide monoethanolamine (CMEA), sodium lauryl sulfate (SLS), and water.

FIG. 3 illustrates a process for the production of the lamellar phase CMEA liquid crystals using an apparatus that has a recirculating cooling loop.

### DETAILED DESCRIPTION OF THE INVENTION

A novel composition containing cocamide monoethanolamine (CMEA) has been discovered that allows the efficient introduction of CMEA into personal care compositions at room temperature or below, using virtually any processing conditions (e.g., batch process, continuous process). Traditionally, the incorporation of CMEA into personal care compositions involved its direct solubilization into micelles, along with detersive surfactants present in the personal care composition, at an elevated temperature. It has been found that if CMEA is first pre-solubilized into an intermediate composition containing micellar or lamellar phase liquid crystals, this composition can easily and efficiently be incorporated into personal care compositions to form personal care products, without the necessity of heat. In one embodiment the composition can be present in the personal care product at a concentration of less than about 30 wt. %, in another embodiment less than 20 wt. %, and in yet another embodiment less than about 12.5 wt. %, based on the total weight of the personal care product.

The ability to form high activity liquid form of CMEA, a detersive surfactant, and water is highly dependent on their relative concentrations. At low concentrations, the CMEA and detersive surfactant are randomly dispersed in water without any ordering. At slightly higher concentrations, these components spontaneously assemble into micelles, which remain unordered in solution. At even higher concentrations, the assemblies become ordered into different phases of liquid crystals. In the hexagonal phase, long cylinders form and are arranged into a hexagonal lattice. In the lamellar phase, the surfactant arranges itself into extended sheets that are separated by thin layers of water. Typical surfactant aggregation schemes can be found in many literature references. One such reference is FIG. 5.8 on page 116 of Laughlin, The Aqueous Phase Behavior of Surfactants, Academic Press: San Diego, Calif. (1994). The liquid crystal phase of the composition described herein includes lamellar phase and lacks a significant hexagonal phase on the dilution path to final personal care product composition. This lack of hexagonal phase during blending allows the micellar or lamellar phase liquid crystals of the composition to be easily blended into micelles upon incorporation into a personal care composition. In contrast, the hexagonal phase is more difficult to disperse or dissolve when incorporated into personal care compositions and typically requires a high energy device such as a mill.

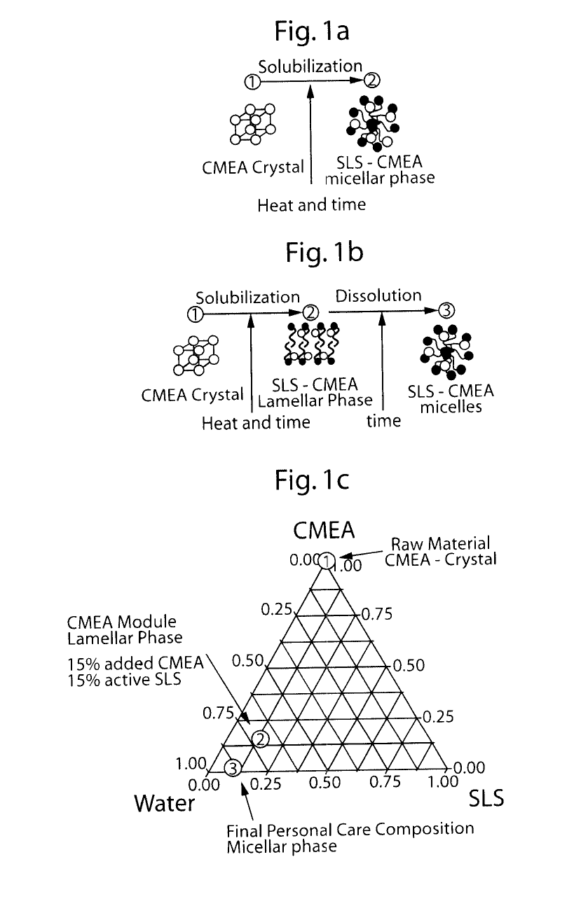

FIG. 1a depicts the traditional method for the solubilization of CMEA into personal care compositions. CMEA crystals (point 1) are introduced into a personal care composition containing a detersive surfactant (e.g., sodium lauryl sulfate (SLS)) and heated. Because this composition is relatively dilute, a micellar phase of CMEA and SLS results over time (point 3). FIG. 1b depicts the intermediate, high activity micellar or lamellar liquid crystal phase composition of the invention and its incorporation into personal care compositions. CMEA crystals (point 1) are introduced to a detersive surfactant (e.g., SLS) and heated. Because this composition is relatively concentrated, a thick micellar or lamellar liquid crystal phase of CMEA and SLS exists (point 2). The composition containing the high activity micellar or lamellar phase liquid crystals of CMEA and SLS can be added to any personal care composition, which results in their dilution to micelles, without the use of heat (point 3). The concentrations of CMEA, surfactant (e.g., SLS) and water at different phases, such as solid CMEA (point 1), lamellar phase liquid crystals (point 2) and micelles (point 3), are depicted in the ternary phase diagram of FIG. 1c.

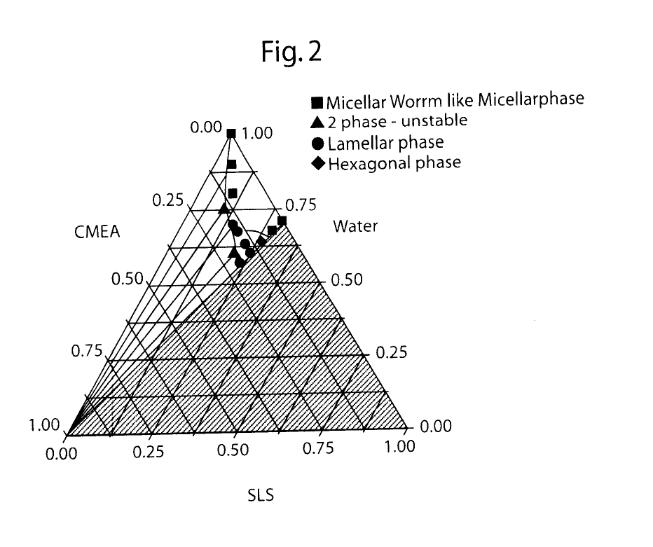

The ability to form an intermediate high CMEA activity micellar or lamellar, liquid crystal phase comprising CMEA and a detersive surfactant in water is highly dependent on the ratio of concentrations of CMEA to detersive surfactant to water. The ternary phase diagram depicted in FIG. 2 shows the concentration window that yields lamellar phase liquid crystals in a CMEA/SLS/water composition at about 23° C.

The concentrations of CMEA, SLS, and water, from 0 wt. % to 100 wt. %, based on the total weight of the composition, are represented by one of the sides of the triangle. Each symbol in the diagram represents the composition at a particular concentration of CMEA, SLS, and water. The squares indicate some specific concentrations of CMEA, SLS, and water when the composition exists in the micellar phase. The circles indicate some specific concentrations of CMEA, SLS, and water when the composition exists in the lamellar, liquid crystal phase. The diamond indicates a specific concentration of CMEA, SLS, and water when the composition exists in the hexagonal, liquid crystal phase. The triangles indicate some specific concentrations of CMEA, SLS, and water when the composition exists in two phases. The boundaries of each of the micellar phase, hexagonal, liquid crystal phase, lamellar, liquid crystal phase, and biphasic are represented by heavy, black lines. As shown in the ternary phase diagram in FIG. 2, the concentration region that allows the composition to exist in the lamellar, liquid crystal phase is quite small, bound by the following component concentrations: about 5 wt. % to about 25 wt % CMEA, about 10 wt. % to about 30 wt. % SLS, and about 55 wt. % to about 85 wt. % water, based on the total weight of the composition.

When the high activity micellar or lamellar phase liquid crystal composition of the invention is added to a personal care composition it undergoes dilution (i.e., the concentration of water increases in relation to the concentrations of CMEA and detersive surfactant). This dilution can be visualized using the ternary phase diagram of FIG. 2. If the initial concentrations of CMEA, SLS, and water in the composition of the invention were represented by the topmost circle, dilution of that particular composition could be represented by drawing a vertical line from the top of the circle towards 100 wt. % water (i.e. towards the top vertex of the triangle). Therefore the composition, which was originally located in the lamellar phase liquid crystal region, crosses the lamellar phase/micellar phase boundary during dilution and directly enters the micellar phase.

As shown in FIG. 2, at some specific concentrations, a single boundary line exists between the lamellar phase and the micellar phase, indicating that the lamellar phase liquid crystals can be directly diluted to micelles. In traditional personal care systems, there is not just one boundary line directly separating the lamellar liquid crystal phase and the micellar phase. Instead, the lamellar liquid crystal phase is surrounded by the hexagonal liquid phase. Thus, diluting a lamellar phase liquid crystal composition into a micellar phase would require the composition to first go through the hexagonal phase before entering the micellar phase. However, diluting hexagonal phase liquid crystals to the micellar phase is extremely difficult, which makes their incorporation into personal care compositions problematic. The fact that the composition of the invention can easily reach the final micellar form when added to a personal care composition is just one of its advantages.

The composition of the invention is also advantageous for the production of personal care compositions. The composition of the invention can be stored at a temperature of about 22° C. to about 30° C. for about three years and incorporated into a personal care composition at will. Further, because the composition is in the micellar or lamellar liquid crystal phase, it does not require heat to form the final micellar structures upon addition to a personal care composition. Therefore, it is useful for heat-sensitive processes or in compositions with heat-sensitive components. Also, the incorporation of the composition of the invention into a personal care composition at room temperature allows faster and more successful scalability when developing a new personal care formulation.

Further still, the ability to add the composition of the invention to a personal care composition at room temperature results in energy and cost savings. For example, traditional methods of solubilizing CMEA into personal care compositions require heating every batch of personal care composition to which CMEA is added. In contrast, the introduction of CMEA into a personal care composition using the high activity micellar or lamellar liquid crystal composition of the invention does not require a heating step. Although the formation of the composition of the invention requires heat, the resulting composition is relatively concentrated and one batch can be used to incorporate CMEA into many different batches of personal care compositions at room temperature or below. The relatively high concentration of the composition of the invention is also advantageous over the traditional, dilute compositions of pre-solubilized CMEA for shipping purposes.

Even further, the composition of the invention can be incorporated into a personal care composition much more quickly than by the traditional method of directly solubilizing CMEA crystals into the personal care composition. This quicker incorporation of CMEA is possible because the high CMEA activity micellar or lamellar phase, liquid crystals are structurally closer to the final micellar phase, which forms when CMEA is added to personal care compositions, than are CMEA crystals.

The term “liquid crystal”, as used herein, means a material having phases that are ordered and/or crystalline in only one or two of their three possible orthogonal directions and are disordered (random and/or liquid-like) in the other dimensions.

The term “polymer”, as used herein, shall include materials whether made by polymerization of one type of monomer or made by two (i.e., copolymers) or more types of monomers.

The term “water soluble”, as used herein, means that the polymer is soluble in water in the present composition. In one embodiment, the polymer should be soluble at 25° C. at a concentration of about 0.1 wt. %, in another embodiment at less than about 1 wt. %, in yet another embodiment at less than about 5 wt. %, and in yet another embodiment at less than about 15 wt. %, based on the weight of the water solvent.

The term “charge density” as used herein, means the ratio of the number of positive charges on a monomeric unit of which a polymer is comprised to the molecular weight of said monomeric unit. The charge density multiplied by the polymer molecular weight determines the number of positively charged sites on a given polymer chain.

The term “alkyl” refers to a saturated or unsaturated straight or branched chain hydrocarbon group of carbon atoms, including, but not limited to, methyl, ethyl, n-propyl, isopropyl, n-butyl, isobutyl, tert-butyl, n-hexyl, and the like. C₁₋₈alkyl refers to substituted or unsubstituted alkyl groups that can have, for example from 1 to 8 carbon atoms. The term “alkyl” includes “bridged alkyl,” i.e., a bicyclic or polycyclic hydrocarbon group, for example, norbornyl, adamantyl, bicyclo[2.2.2]octyl, bicyclo[2.2.1]heptyl, bicyclo[3.2.1]octyl, or decahydronaphthyl. Alkyl groups optionally can be substituted, for example, with hydroxy (OH), halogen, aryl, heteroaryl, cycloalkyl, heterocycloalkyl, and amino. “Heteroalkyl” is defined similarly as alkyl, except the heteroalkyl contains at least one heteroatom independently selected from the group consisting of oxygen, nitrogen, and sulfur.

The term “alkylene” refers to a straight or branched alkyl group chain having two points of attachment to the rest of the molecule.

The term “alkenyl” refers to a straight or branched chain hydrocarbon group of at least two carbon atoms containing at least one carbon double bond including, but not limited to, 1-propenyl, 2-propenyl, 2-methyl-1-propenyl, 1-butenyl, 2-butenyl, and the like.

The term “alkylidene” refers to a straight or branched alkene group having two points of attachment to the rest of the molecule.

The term “alkoxy” refers to a straight or branched chain alkyl group covalently bonded to the parent molecule through an —O— linkage. Examples of alkoxy groups include, but are not limited to, methoxy, ethoxy, propoxy, isopropoxy, butoxy, n-butoxy, sec-butoxy, t-butoxy and the like.

The term “oxyalkylene” refers to an alkoxy group having two points of attachment to the rest of the molecule, one of the points being through the oxygen atom.

The term “alkoxyalkyl” refers to one or more alkoxy groups appended to an alkyl group.

The term “aryl” refers to a monocyclic or polycyclic aromatic group, in one embodiment a monocyclic or bicyclic aromatic group, e.g., phenyl or naphthyl. Unless otherwise indicated, an aryl group can be unsubstituted or substituted with one or more, and in particular one to five groups independently selected from, for example, halogen, alkyl, alkenyl, OCF₃, NO₂, CN, NC, OH, alkoxy, amino, CO₂H, CO₂alkyl, aryl, and heteroaryl. Exemplary aryl groups include, but are not limited to, phenyl, naphthyl, tetrahydronaphthyl, chlorophenyl, methylphenyl, methoxyphenyl, trifluoromethylphenyl, nitrophenyl, 2,4-methoxychlorophenyl, and the like.

The term “heteroaryl” refers to a monocyclic or polycyclic aromatic group, in one embodiment a monocyclic or bicyclic aromatic group, containing at least one nitrogen, oxygen, or sulfur atom in an aromatic ring. Unless otherwise indicated, a heteroaryl group can be unsubstituted or substituted with 1 to 5 groups. Examples of heteroaryl groups include, but are not limited to, thienyl, furyl, pyridyl, oxazolyl, quinolyl, thiophenyl, isoquinolyl, indolyl, triazinyl, triazolyl, isothiazolyl, isoxazolyl, imidazolyl, benzothiazolyl, pyrazinyl, pyrimidinyl, thiazolyl, and thiadiazolyl.

The term “alkylaryl” refers to one or more alkyl groups appended to an aryl group.

The term “alkoxyaryl” refers to one or more alkoxy groups appended to an aryl group.

The term “arylalkyl” refers to one or more aryl groups appended to an alkyl group.

The term “aryloxy” refers to an aromatic group covalently bonded to the parent molecule through an —O— linkage.

The term “alkylaryloxy” refers to an alkylaryl group covalently bonded to the parent molecule through an —O— linkage.

The term “alkanol” refers to a straight or branched chain alkyl group covalently bonded to OH.

The term “alkanolamine” refers to straight or branched chain alkyl groups covalently bonded to a hydroxy moiety and to a amino moiety. Examples of alkanolamine include propanolamine, ethanolamine, dimethylethanolamine, and the like.

The term “amido” refers to a group having a NH₂ radical that is bonded to a C═O radical.

The term “alkanolamide” refers to a straight or branched chain alkyl group covalently bonded to a hydroxy moiety and to an amide moiety.

The term “alkylsulfate” refers to a straight or branched chain alkyl group covalently to SO₃⁻.

The term “benzyl” refers to a benzene radical that can be unsubstituted or substituted with one or more, and in particular one to five groups independently selected from, for example, halogen, alkyl, alkenyl, OCF₃, NO₂, CN, NC, OH, alkoxy, amino, CO₂H, CO₂alkyl, aryl, and heteroaryl.

The term “halogen” or “halo” refers to fluoro, chloro, bromo, or iodo.

All percentages, parts and ratios are based upon the total weight of the compositions of the present invention, unless otherwise specified. All such weights as they pertain to listed ingredients are based on the active level and therefore do not include solvents or by-products that may be included in commercially available materials, unless otherwise specified. The term “weight percent” may be denoted as “wt. %” herein.

All molecular weights as used herein are weight average molecular weights expressed as grams/mole, unless otherwise specified.

In one aspect, the invention relates to a composition that contains high CMEA activity micellar or lamellar phase liquid crystals of cocamide monoethanolamine (CMEA) and a detersive surfactant in water. In some embodiments, the composition of the invention is at a temperature range of about 20° C. to about 30° C. The composition of the invention is phase stable above about 17° C., meaning that CMEA and/or SLS do not crystallize out of solution above this temperature.

The composition of the invention has a molar ratio of detersive surfactant to CMEA to water that allows it to exist in the lamellar liquid crystal phase. In some embodiments, the molar ratio of CMEA to detersive surfactant about 1:1 to about 1:20, while the molar ratio of CMEA to water is about 1:50 to about 1:1000. For example the molar ratio of CMEA to detersive surfactant to water can be about 1:1:50 to about 1:20:50 or about 1:1:1000 to about 1:20:1000. In some embodiments, the molar ratio of detersive surfactant to CMEA is about 1:1 to about 1:5, while the molar ratio of CMEA to water is about 1:50 to about 1:250. For example the molar ratio of CMEA to detersive surfactant to water can be about 1:1:50 to about 1:5:50 or about 1:1:250 to about 1:5:250. The molar ratio of CMEA to detersive surfactant to water can be about 1:2:150. Expressed another way, the composition of the invention can include about 5 wt. % to about 25 wt. % of CMEA, about 10 wt. % to about 30 wt. % of detersive surfactant, and about 55 wt. % to about 85 wt. % of water, based on the total weight of the composition.

The composition of the invention includes a detersive surfactant. The detersive surfactant provides cleaning performance to the composition of the invention and aids in the formation of the lamellar liquid crystal phase. The detersive surfactant contains at least one anionic surfactant, which has an ethyoxylate level of about 0 to about 10 and an anion level of about 1 to about 10, and optionally an amphoteric surfactant, a zwitterionic surfactant, a cationic surfactant, a nonionic surfactant, or a combination thereof. Such surfactants should be physically and chemically compatible with the essential components described herein, or should not otherwise unduly impair product stability, aesthetics, or performance.

An optimal ethoxylate level is calculated based on the stoichiometry of the surfactant structure, which in turn is based on a particular molecular weight of the surfactant where the number of moles of ethoxylation is known. Likewise, given a specific molecular weight of a surfactant and an anionization reaction completion measurement, the anion level can be calculated. Analytical techniques have been developed to measure ethoxylation or anionization within surfactant systems.

The Level of Ethoxylate and the Level of Anion representative of a particular surfactant system are calculated from the percent ethoxylation and percent anion of individual surfactants in the following manner. The Level of Ethoxylate is equal to the percent ethoxylation multiplied by percent active ethoxylated surfactant (based on the total weight of the composition). The Level of Anion is equal to the percent anion in ethyoxylated surfactant multiplied by percent active ethoxylated surfactant (based on the total weight of the composition) plus percent anion in non-ethoxylated surfactant (based on the total weight of the composition). If a composition comprises two or more surfactants having different respective anions (e.g., surfactant A has a sulfate group and surfactant B has a sulfonate group), the Level of Anion in the composition is the sum of the molar levels of each respective anion as calculated above.

For example, a detersive surfactant contains 48.27 wt. % sodium laureth sulfate 3-ethoxylate (SLE3S) and 6.97 wt. % sodium lauryl sulfate (SLS), based on the total weight of the composition. The ethoxylated surfactant (SLE3S) contains 0.294321% ethoxylate and 0.188307% sulfate as the anion and the non-ethoxylated surfactant (SLS) contains 0.266845% sulfate as an anion. Because both of the SLE and SLS are about 29% active, the detersive surfactant contains about 14 wt. % of active SLE3S and about 2 wt. % of active SLS, based on the total weight of the composition. The Level of Ethoxylate is 0.294321 multiplied by 14 (% active ethoxylated surfactant). Thus, the Level of Ethoxylate in this example detersive surfactant is 4.12. The Level of Anion is 0.188307 multiplied by 14 (% active ethoxylated surfactant) plus 0.266845 multiplied by 2 (% active non-ethoxylated surfactant). Thus the Level of Anion in this example detersive surfactant is 3.17.

In one embodiment, the detersive surfactant includes at least one anionic surfactant that contains an anion selected from the group consisting of sulfates, sulfonates, sulfosuccinates, isethionates, carboxylates, phosphates, and phosphonates. In one embodiment the anion is a sulfate. Other potential anions for the anionic surfactant include phosphonates, phosphates, and carboxylates.

Anionic surfactants suitable for use in the personal care compositions are alkyl sulfates and alkyl ether sulfates. These materials have the respective formulae ROSO₃M and RO(C₂H₄O)ₓSO₃M, wherein R is alkyl or alkenyl of about 8 to about 18 carbon atoms, x is an integer having a value of about 1 to about 10, and M is a cation such as ammonium, an alkanolamine such as triethanolamine, a monovalent metal cation such as sodium and potassium, or a polyvalent metal cation such as magnesium and calcium. Solubility of the surfactant will depend upon the particular anionic surfactants and cations chosen.

In one embodiment, R has about 8 to about 18 carbon atoms, in another embodiment from about 10 to about 16 carbon atoms, in yet another embodiment from about 12 to about 14 carbon atoms, in both the alkyl sulfates and alkyl ether sulfates. The alkyl ether sulfates are typically made as condensation products of ethylene oxide and monohydric alcohols having about 8 to about 24 carbon atoms. The alcohols can be synthetic or they can be derived from fats, e.g., coconut oil, palm kernel oil, tallow. Lauryl alcohol and straight chain alcohols derived from coconut oil or palm kernel oil can be used. In one embodiment such alcohols are reacted with about 0 to about 10, in another embodiment from about 0 to about 5, and in yet another embodiment from about 0, 1 or 3, molar proportions of ethylene oxide, and the resulting mixture of molecular species having, for example, an average of 0, 1, or 3 moles of ethylene oxide per mole of alcohol is sulfated and neutralized.

Specific non-limiting examples of alkyl ether sulfates which may be used in the composition of the invention include sodium and ammonium salts of coconut alkyl triethylene glycol ether sulfate, tallow alkyl triethylene glycol ether sulfate, and tallow alkyl hexa-oxyethylene sulfate. Suitable alkyl ether sulfates are those comprising a mixture of individual compounds, wherein the compounds in the mixture have an average alkyl chain length of about 10 to about 16 carbon atoms and an average degree of ethoxylation of about 1 to about 4 moles of ethylene oxide. Such a mixture also comprises about 0 wt. % to about 20 wt. % of C₁₂₋₁₃ compounds; about 60 wt. % to about 100 wt. % of C₁₄₋₁₆ compounds; about 0 wt. % to about 20 wt. % of C₁₇₋₁₉ compounds; about 3 wt. % to about 30 wt. % of compounds having a degree of ethoxylation of 0; about 45 wt. % to about 90 wt. % of compounds having a degree of ethoxylation about 1 to about 4; about 10 wt. % to about 25 wt. % of compounds having a degree of ethoxylation of about 4 to about 8; and about 0.1 wt. % to about 15 wt. % by weight of compounds having a degree of ethoxylation greater than about 8, based on the total weight of the alkyl ether sulfate.

Suitable anionic detersive surfactant components include those which are known for use in hair care or other personal care cleansing compositions. Suitable anionic detersive surfactants components for use in the composition of the invention include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, and combinations thereof.

In some embodiments, the detersive surfactant further includes one or more additional surfactants selected from the group consisting of amphoteric surfactants, zwitterionic surfactants, cationic surfactants, nonionic surfactants, and mixtures thereof. These surfactants are known for use in hair care or other personal care cleansing compositions and contain a group that is anionic at the pH of the composition. The concentration of such amphoteric detersive surfactants in these personal cleansing compositions ranges from about 0.5 wt. % to about 20 wt. %, in another embodiment from about 1 wt. % to about 10 wt. %, based on the total weight of the cleansing composition. Non-limiting examples of suitable zwitterionic or amphoteric surfactants are described in U.S. Pat. Nos. 5,104,646 and 5,106,609.

Amphoteric surfactants suitable for use in the composition of the invention are well known in the art, and include those surfactants broadly described as derivatives of aliphatic secondary and tertiary amines in which the aliphatic radical can be straight or branched chain and wherein one of the aliphatic substituents contains from about 8 to about 18 carbon atoms and one contains an anionic water solubilizing group such as carboxy, sulfonate, sulfate, phosphate, or phosphonate. Suitable amphoteric surfactants for use in the personal care compositions comprise cocoamphoacetate, cocoamphodiacetate, lauroamphoacetate, lauroamphodiacetate, lauramine oxide, and mixtures thereof.

Zwitterionic surfactants suitable for use in the personal care composition are well known in the art, and include those surfactants broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group such as carboxy, sulfonate, sulfate, phosphate or phosphonate. Zwitterionics such as betaines (i.e., cocoamidopropyl betaine, cocobetaine), are useful herein.

The composition of the invention may further comprise additional surfactants for use in combination with the detersive surfactant component described herein. Other suitable anionic surfactants are the water-soluble salts of organic, sulfuric acid reaction products conforming to the formula [R¹—SO₃-M] where R¹ is a straight or branched chain, saturated, aliphatic hydrocarbon radical having from about 8 to about 24, in one embodiment from about 10 to about 18 carbon atoms; and M is a cation, as previously described herein. Nonlimiting examples of such surfactants are the salts of an organic sulfuric acid reaction product of a hydrocarbon of the methane series, including iso-, neo-, and n-paraffins, having from about 8 to about 24 carbon atoms, in one embodiment from about 12 to about 18 carbon atoms and a sulfonating agent, e.g., SO₃, H₂SO₄, obtained according to known sulfonation methods, including bleaching and hydrolysis. Also suitable for use herein are alkali metal and ammonium sulfonated C₁₀₋₁₈ n-paraffins.

Other suitable anionic surfactants are the reaction products of fatty acids esterified with isethionic acid and neutralized with sodium hydroxide where, for example, the fatty acids are derived from coconut oil or palm kernel oil; sodium or potassium salts of fatty acid amides of methyl tauride in which the fatty acids, for example, are derived from coconut oil or palm kernel oil.

Other anionic surfactants suitable for use in the composition of the invention are the succinates, examples of which include disodium N-octadecylsulfosuccinate; disodium lauryl sulfosuccinate; diammonium lauryl sulfosuccinate; tetrasodium N-(1,2-dicarboxyethyl)-N-octadecylsulfosuccinnate; diamyl ester of sodium sulfosuccinic acid; dihexyl ester of sodium sulfosuccinic acid; and dioctyl esters of sodium sulfosuccinic acid.

Other suitable anionic surfactants include olefin sulfonates having about 10 to about 24 carbon atoms. In this context, the term “olefin sulfonates” refers to compounds which can be produced by the sulfonation of alpha-olefins by means of uncomplexed sulfur trioxide, followed by neutralization of the acid reaction mixture in conditions such that any sulfonates which have been formed in the reaction are hydrolyzed to give the corresponding hydroxy-alkanesulfonates. The sulfur trioxide can be liquid or gaseous, and is usually, but not necessarily, diluted by inert diluents, for example by liquid SO₂, chlorinated hydrocarbons, etc., when used in the liquid form, or by air, nitrogen, gaseous SO₂, etc., when used in the gaseous form. The alpha-olefins from which the olefin sulfonates are derived are mono-olefins having about 10 to about 24 carbon atoms, and in one embodiment from about 12 to about 16 carbon atoms. In one embodiment, they are straight chain olefins. In addition to the true alkene sulfonates and a proportion of hydroxy-alkanesulfonates, the olefin sulfonates can contain minor amounts of other materials, such as alkene disulfonates depending upon the reaction conditions, proportion of reactants, the nature of the starting olefins and impurities in the olefin stock and side reactions during the sulfonation process. A nonlimiting example of such an alpha-olefin sulfonate mixture is described in U.S. Pat. No. 3,332,880.

Another class of anionic surfactants suitable for use in the compositions of the invention are the beta-alkyloxy alkane sulfonates. These surfactants conform to the Formula I:

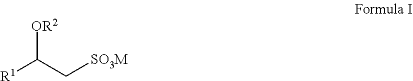

where R¹ is a straight chain alkyl group having about 6 to about 20 carbon atoms, R² is a lower alkyl group having about 1 to about 3 carbon atoms, and in one embodiments 1 carbon atom, and M is a water-soluble cation, as previously described herein. Suitable anionic surfactants for use in the composition of the invention include sodium tridecyl benzene sulfonate, sodium dodecyl benzene sulfonate, and mixtures thereof.

Amides, including alkanolamides, are the condensation products of fatty acids with primary and secondary amines or alkanolamines to yield products of the general Formula II:

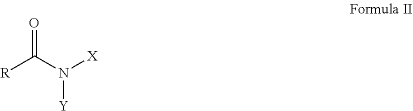

wherein RCO is a fatty acid radical and R is C₈₋₂₀; X is an alkyl, aromatic or alkanol (CHR′CH₂OH wherein R′ is H or C₁₋₆ alkyl); Y is H, alkyl, alkanol or X. Suitable amides include, but are not limited to cocamide, lauramide, oleamide and stearamide. Suitable alkanolamides include, but are not limited to, cocamide DEA, cocamide MEA, cocamide MIPA, isostearamide DEA, isostearamide MEA, isostearamide MIPA, lanolinamide DEA, lauramide DEA, lauramide MEA, lauramide MIPA, linoleamide DEA, linoleamide MEA, linoleamide MIPA, myristamide DEA, myristamide MEA, myristamide MIPA, Oleamide DEA, Oleamide MEA, Oleamide MIPA, palmamide DEA, palmamide MEA, palmamide MIPA, palmitamide DEA, palmitamide MEA, palm kernelamide DEA, palm kernelamide MEA, palm kernelamide MIPA, peanutamide MEA, peanutamide MIPA, soyamide DEA, stearamide DEA, stearamide MEA, stearamide MIPA, tallamide DEA, tallowamide DEA, tallowamide MEA, undecylenamide DEA, undecylenamide MEA, PPG-2 Hydroxyethyl cocoamide, and PPG-2-Hydroxyethyl Coco/Isostearamide. The condensation reaction may be carried out with free fatty acids or with all types of esters of the fatty acids, such as fats and oils, and particularly methyl esters. The reaction conditions and the raw material sources determine the blend of materials in the end product and the nature of any impurities.

Suitable optional surfactants include nonionic surfactants. Any such surfactant known in the art for use in hair or personal care products may be used, provided that the optional additional surfactant is also chemically and physically compatible with the essential components of the composition of the invention, or does not otherwise unduly impair product performance, aesthetics or stability. The concentration of the optional additional surfactants in the personal care composition may vary with the cleansing or lather performance desired, the optional surfactant selected, the desired product concentration, the presence of other components in the composition, and other factors well known in the art.

Nonlimiting examples of other surfactants suitable for use in the personal care compositions are described in McCutcheon's, Emulsifiers and Detergents, 1989 Annual, published by M.C. Publishing Co., and U.S. Pat. Nos. 3,929,678; 2,658,072; 2,438,091; 2,528,378.

In some embodiments of the invention, the detersive surfactant includes sodium lauryl sulfate (SLS). For example, the composition of the invention can include about 6.8 wt. % to about 15 wt. % of CMEA, about 12 wt. % to about 22 wt. % of sodium lauryl sulfate, and about 62.5 wt. % to about 81.2 wt % of water, based on the total weight of the composition. In one embodiment, the composition can include about 15 wt. % of CMEA, about 12.5 wt. % of sodium lauryl sulfate, and about 72.5 wt. % of water, based on the total weight of the composition.

In some embodiments of the invention, the detersive surfactant includes sodium laureth sulfate 1-ethoxylate (SLE1S). For example, the composition of the invention can include about 6.8 wt. % to about 15 wt. % of CMEA, about 12 wt. % to about 22 wt. % of SLE1S, and about 62.5 wt. % to about 81.2 wt % of water, based on the total weight of the composition. The composition can include about 15 wt. % of CMEA, about 12.5 wt. % of SLE1S, and about 72.5 wt. % of water, based on the total weight of the composition.

In some embodiments of the invention, the detersive surfactant includes sodium laureth sulfate 3-ethoxylate (SLE3S). For example, the composition of the invention can include about 6.8 wt. % to about 15 wt. % of CMEA, about 12 wt. % to about 22 wt. % of SLE3S, and about 62.5 wt. % to about 81.2 wt % of water, based on the total weight of the composition. The composition can include about 15 wt. % of CMEA, about 12.5 wt. % of SLE3S, and about 72.5 wt. % of water, based on the total weight of the composition.

The composition of the invention can further include a hydrotrope. The hydrotrope lowers the surfactant order structure, increasing the effective ratio of CMEA to detersive surfactant. The hydrotrope can be any hydrotrope used in the personal care industry, including short chain surfactants that help to solubilize other surfactants. In some embodiments, the hydrotrope includes C₁₋₈alkyl carboxylates, C₁₋₈alkyl sulfates, C₁₋₈alkyl benzene sulfonates, halogen benzoates (e.g., chlorbenzoate), C₁₋₈alkyl naphthalene carboxylates, (e.g., hydroxyl naphthalene carboxylate), urea, ethoxylated sulfates, and mixtures thereof. The C₁₋₈alkyl benzene sulfonates can include C₁₋₈alkyl cumene sulfonates, C₁₋₈alkyl toluene sulfonates (e.g., para-toluene sulfonate), C₁-C₈alkyl xylene sulfonates, and mixtures thereof. For example, the hydrotrope can include, sodium xylene sulfonates, potassium xylene sulfonates, ammonium xylene sulfonates, calcium xylene sulfonates, sodium toluene sulfonates, potassium toluene sulfonates, sodium cumene sulfonates, ammonium cumene sulfonates, sodium alkyl naphthalene sulfonates, sodium butyl naphthalene sulfonates, and mixtures thereof. The hydrotrope can be present in any amount that is sufficient to help solubilize CMEA. In some embodiments, the hydrotrope is present in an amount of about 0.5 wt. % to about 5.0 wt. %, about 1.0 wt. % to about 3.0 wt. %, based on the total weight of the composition.

The composition of the invention can further include an electrolyte. The electrolyte raises the surfactant order structure, and decreases the effective ratio of detersive surfactant to CMEA. The electrolyte can be an inorganic salt or an organic salt. Generally, inorganic electrolytes are preferred over organic electrolytes for better weight efficiency and lower costs. Mixtures of inorganic and organic salts can be used. Typical levels of electrolyte in the compositions are less than about 10 wt. %, based on the total weight of the composition. In one embodiment, the level of electrolytes in the composition is about 0.5 wt. % to about 5 wt. % by weight, based on the total weight of the composition, in another embodiment from about 0.75 wt. % to about 2.5 wt. %, and in yet another embodiment from about 1 wt. % to about 2 wt. %, based on the total weight of the composition.

Nonlimiting examples of inorganic salts suitable for use in the composition of the invention include MgI₂, MgBr₂, MgCl₂, Mg(NO₃)₂, Mg₃(PO₄)₂, Mg₂P₂O₇, MgSO₄, magnesium silicate, Ng, NaBr, NaCl, NaF, Na₃(PO₄), NaSO₃, Na₂SO₄, Na₂SO₃, NaNO₃, NaIO₃, Na₃(PO₄), Na₄P₂O₇, sodium silicate, sodium metasilicate, sodium tetrachloroaluminate, sodium tripolyphosphate (STPP), Na₂Si₃O₇, sodium zirconate, CaF₂, CaCl₂, CaBr₂, CaI₂, CaSO₄, Ca(NO₃)₂, Ca, KI, KBr, KCl, KF, KNO₃, KIO₃, K₂SO₄, K₂SO₃, K₃(PO₄), K₄(P₂O₇), potassium pyrosulfate, potassium pyrosulfite, LiI, LiBr, LiCl, LiF, LiNO₃, AlF₃, AlCl₃, AlBr₃, AlI₃, Al₂(SO₄)₃, Al(PO₄), A(NO₃)₃, aluminum silicate; including hydrates of these salts and including combinations of these salts or salts with mixed cations e.g. potassium alum AlK(SO₄)₂ and salts with mixed anions, e.g. potassium tetrachloroaluminate and sodium tetrafluoroaluminate. Mixtures of above salts are also useful

Organic salts useful in this invention include, magnesium, sodium, lithium, potassium, zinc, and aluminum salts of the carboxylic acids including formate, acetate, proprionate, pelargonate, citrate, gluconate, lactate aromatic acids e.g., benzoates, phenolate and substituted benzoates or phenolates, such as phenolate, salicylate, polyaromatic acids terephthalates, and polyacids e.g., oxylate, adipate, succinate, benzenedicarboxylate, benzenetricarboxylate. Other useful organic salts include carbonate and/or hydrogencarbonate (HCO₃⁻¹) when the pH is suitable, alkyl and aromatic sulfates and sulfonates e.g., sodium methyl sulfate, benzene sulfonates and derivatives such as xylene sulfonate, and amino acids when the pH is suitable. Electrolytes can comprise mixed salts of the above, salts neutralized with mixed cations such as potassium/sodium tartrate, partially neutralized salts such as sodium hydrogen tartrate or potassium hydrogen phthalate, and salts comprising one cation with mixed anions.

The composition of the invention can further include a preservative. The preservative is an antimicrobial substance that kills or inhibits the growth of microorganisms such as bacteria, fungi, or protozoans. Nonlimiting examples of the preservative include sodium benzoate, benzyl alcohol, potassium sorbate, disodium ethylenediamine tetraacetate (Na₂EDTA), tetrasodium ethylenediamine tetraacetate (Na₄EDTA), methylchloroisothiazolinone (KATHON®), and mixtures thereof. The preservative can be present in any amount that is effective for killing or inhibiting the growth of microorganisms. The amount of preservative is dependent on the specific preservative used. For example, the preservative can include at least about 0.25 wt. % sodium benzoate, about 5 ppm to about 15 ppm of methylchloroisothiazolinone (KATHON®), or mixtures thereof, based on the total weight of the composition. Additional examples of preservatives useful for the composition of the invention and suitable amounts (based on the total weight of the composition) are listed in Table 1.

TABLE 1
Preservatives

| | | Amount (based on the total |
|---|---|---|
| Class | Name | weight of a composition) |
| Formaldehyde | Formaldehyde | 0.05 wt. % to 0.2 wt. % |
| and/or | Germall 115 imidazolidinyl urea | 0.05 wt. to 0.5 wt. % |
| Formaldehyde | [N-methylenebis-(N-1-(hydroxymethyl)- | |
| Donors | 2,5-dioxo-4-imidazolidinyl) urea] | |
| | Germall II diazolidinyl urea | 0.03 wt. % to 0.3 wt. % |
| | [N-(hydroxymethyl)-N-(1,3- | |
| | dihydroxymethyl-2,5-dioxo-4- | |
| | imidazolidinyl-n(hydroxymethyl) urea] | |
| | DOWICIL ® 200 (Quaterium 15) | 0.02 wt. % to 0.3 wt. % |
| | [cis-isomer of 1-(3-chloroallyl)-3,5,7- | |
| | triaza-1-azoniaadamantane chloride] | |
| | GLYDANT ® (1,3-Bis(hydroxymethyl)- | 0.15 wt. % to 0.4 wt. % |
| | 5,5-dimethylhydantoin) or other | |
| | dimethylol dimethyl hydantoin | |
| | (DMDMH) actives | |
| | Glutaraldehyde | 0.01 wt. % to 0.1 wt. % |
| | | (active) |
| | Bronopol 2-bromo-2-nitropropane-1,3 | 0.01 wt. % to 0.1 wt. % |
| | diol | |
| Parabens | Hydroxybenzoates (methyl, ethyl, | less than or equal to 0.3 |
| | propyl, butyl, isobutyl) | wt. % |
| Isothiazolinones | 2-Methyl-4-isothiazoline-3-one | 50 ppm to 100 ppm (active) |
| | 5-Chloro-2-methyl-4-isothiazoline-3-one | typically used in |
| | | combination with 2-methyl- |
| | | 4-isothiazoline-3-one |
| | Isothiazolinone | 5 ppm to 15 ppm (active), |
| | | 0.033 wt. % to 0.1 wt. % (as |
| | | added) |
| Organic acids | Dehydroacetic acid (3-acetyl-6-methyl- | 0.02 wt. % to 0.2 wt. % |
| | 2H-pyran-2,4-(3H)-dione) and its salts | |
| | Sorbic acid (2,4-hexadienoic acid) and | 0.025 wt. % to 0.2 wt. % |
| | its salts | |
| | Benzoic acid and its salts | 0.1 wt. % to 0.2 wt. % |
| | Citric acid and salts | amount necessary to reach |
| | | pH |
| | Propionic acid and salts | up to 1 wt. % |
| | Boric acid | 0.01 wt. % to 1 wt. % |
| | Salicylic acid (ortho-hydroxybenzoic | 0.1 wt. % to 0.5 wt. % |
| | acid) | |
| Alcohols | Benzyl alcohol | 0.25 wt. % 3 wt. % |
| | Chlorobutanol | up to 0.5 wt. %% |
| | Dichlorobenzyl alcohol | 0.05 wt. % to 0.5 wt. % |
| | Phenethyl alcohol (2-phenylethanol) | 1 wt. % to 2 wt. % |
| | 2-Phenoxyethanol | 0.5 wt. % 1 wt. % |
| | Ethanol | 10 wt. % or more |
| Phenolics | Chloroxylenol (para-chloro-2,5- | 0.2 wt. % to 0.8 wt. % |
| | dimethylphenol) | |
| | ortho-Phenylphenol | 0.05 wt. % to 0.5 wt. % |
| Pyrithiones | Zinc pyrithione (zinc-bis-(2- | 250 ppm to 1000 ppm |
| | pyridinethiol-2-oxide) | (active) |
| | Sodium pyrithione (sodium 2- | 250 ppm to 1000 ppm |
| | pyridinethiol-1-oxide) | (active) |

The composition of the invention can further include an acid. The acid functions to neutralize the composition to a pH of about 3 to about 9, in one embodiment from about 4 to about 8. Nonlimiting examples of the acid include hydrochloric acid, citric acid, aspartic acid, glutamic acid, carbonic acid, tatronic acid, malic acid, malonic acid, tartaric acid, adipic acid, phosphoric acid, phthalic acid, glycolic acid, lactic acid, succinic acid, acetic acid, sulfuric acid, boric acid, formic acid, and mixtures thereof. The acid is present in any amount that results in the desired pH. For example, about 0.5 wt. % to about 1.5 wt. % of 6N HCl can be included in the composition of the invention.

The composition of the invention can further include an additive. Nonlimiting examples of the additive can include: conditioning agents, natural cationic deposition polymers, synthetic cationic deposition polymers, anti-dandruff agents, gel networks (e.g., fatty alcohol/surfactant networks), particles, suspending agents, paraffinic hydrocarbons, propellants, viscosity modifiers, dyes, non-volatile solvents, water soluble diluents, water insoluble diluents, pearlescent aids, foam boosters, additional surfactants or nonionic cosurfactants, pediculocides, pH adjusting agents, perfumes, preservatives, chelants, proteins, skin active agents, sunscreens, UV absorbers, vitamins, and mixtures thereof. The concentration of the one or more additives is dependent on the specific additive used and is typically at any concentration traditionally used for the additive in the personal care industry, as elsewhere described herein.

In another aspect, the invention relates to a method for preparing a composition that contains high CMEA activity micellar or lamellar phase liquid crystals of coconut monoethanol amide (CMEA) and a detersive surfactant in water. The composition of the invention is phase stable above about 17° C., meaning that CMEA and/or SLS do not crystallize out of solution above this temperature. In this method, cocamide monoethanolamine (CMEA) is added to a solution containing a neutralized, preserved detersive surfactant at a temperature above the melting point of CMEA (e.g., about 50° C. to about 86° C., in one embodiment from about 60° C. to about 70° C.) to form a composition comprising lamellar phase liquid crystals, which is then cooled to about 22° C. to about 85° C., in one embodiment from about 27° C. to about 33° C.

As previously described, the detersive surfactant contains an anionic surfactant, which has an ethyoxylate level from about 0 to about 10 and an anion level from about 1 to about 10, and should be physically and chemically compatible with the essential components described herein or should not otherwise unduly impair product stability, aesthetics, or performance. Suitable examples of the detersive surfactant are as previously described herein.

In some embodiments, the detersive surfactant can further include amphoteric surfactants, zwitterionic surfactants, cationic surfactant, nonionic surfactants, or mixtures thereof for use in combination with the anionic detersive surfactant component, as previously described herein. Suitable examples of these optional surfactants are as previously described herein.

The relative concentrations of the components (e.g., CMEA, detersive surfactant, water) in the composition of the invention are any concentrations that allow the composition of the invention to exist in the lamellar, liquid crystal phase, as previously described herein.

In some embodiments of this aspect of the invention, the neutralized, preserved, detersive surfactant can be prepared by: (i) adding a detersive surfactant to water; (ii) adding a preservative to the detersive surfactant to form a preserved, detersive surfactant; and (iii) adding acid to the preserved, detersive surfactant to form a neutralized, preserved, detersive surfactant. The preservative and acid can be added to the detersive surfactant at any rate and at a temperature of about 20° C. to about 99° C., in one embodiment from about 20° C. to about 75° C.

The acid functions to neutralize the composition to a pH of about 3 to about 9, in one embodiment from about 4 to about 8, and is present in any amount that results in the desired pH. Nonlimiting examples of the acid are as previously described herein.

As previously described herein, CMEA is added to a solution containing a neutralized, preserved, detersive surfactant at a temperature above the melting point of CMEA (e.g., about 54° C. to about 86° C., in one embodiment from about 60° C. to about 70° C.) to form a composition comprising lamellar phase liquid crystals. In some embodiments, the neutralized, preserved surfactant is prepared at room temperature and then heated during the introduction of the CMEA. In alternative embodiments, the neutralized, preserved surfactant is prepared at an elevated temperature, and kept at the elevated temperature during addition of the CMEA. In other alternative embodiments, the neutralized, preserved surfactant and the CMEA are both preheated before introduction of the CMEA to the neutralized, preserved surfactant.

In some embodiments, the detersive surfactant is subjected to agitation during addition of water, preservative, acid, and/or CMEA. Agitation occurs at a rate sufficient to ensure good homogenization without causing aeration. The exact rate of agitation depends on the size and type of vessel that houses the detersive surfactant. For example, the rate of agitation during addition of the detersive surfactant to water when using a 130 L double impeller baffled tank can be about 100 rpm, while the rate of agitation when using a 15 L double impeller baffled tank can be about 180 rpm. The rate of agitation during addition of a preservative or acid to the detersive surfactant can be, for example, about 40 to about 50 rpm when using a 130 L double impeller baffled tank. The rate of agitation of the detersive surfactant during the addition of CMEA is typically at least twice the rate of agitation of the detersive surfactant during addition of the preservative or acid. Introducing CMEA to the detersive surfactant increases the viscosity of the composition and, thus, needs a faster rate of agitation.

In another aspect, the invention relates to a method of making a personal care product. In this method, the composition of the invention is combined with a personal care composition that includes one or more of the following: conditioning agents, natural cationic deposition polymers, synthetic cationic deposition polymers, anti-dandruff agents, gel networks (e.g., fatty alcohol/surfactant networks), particles, suspending agents, paraffinic hydrocarbons, propellants, viscosity modifiers, dyes, non-volatile solvents, water soluble diluents, water insoluble diluents, pearlescent aids, foam boosters, additional surfactants or nonionic cosurfactants, pediculocides, pH adjusting agents, perfumes, preservatives, chelants, proteins, skin active agents, sunscreens, UV absorbers, vitamins, and mixtures thereof, to form a personal care product.

The composition of the invention can be added to the personal care composition at a concentration that will result in about 0.025 wt. % to about 5 wt. %, in one embodiment from about 0.5 wt. % to about 2 wt. %, in another embodiment from about 0.75 wt. % to about 1.0 wt. % of CMEA, based on the total weight of the personal care product. For example, about 3.33 wt. % to about 13.2 wt. %, and in one embodiment from about 5 wt. % to about 10 wt %, of the composition of the invention can be added to a personal care composition to form a personal care product, based on the total weight of the personal care product.

The composition of the invention can be added to the personal care composition by any means or method typically used to make personal care products.

### 1. Conditioning Agents

a. Oily Conditioning Agent

In some embodiments, the composition of the invention is combined with a personal care composition that includes one or more oily conditioning agents to form a personal care product. Oily conditioning agents include materials which are used to give a particular conditioning benefit to hair and/or skin. In hair treatment compositions, suitable conditioning agents are those which deliver one or more benefits relating to shine, softness, combability, antistatic properties, wet-handling, damage, manageability, body, and greasiness. The oily conditioning agents useful in the compositions of the present invention typically comprise a water-insoluble, water-dispersible, non-volatile, liquid that forms emulsified, liquid particles. Suitable oily conditioning agents for use in the composition are those conditioning agents characterized generally as silicones (e.g., silicone oils, cationic silicones, silicone gums, high refractive silicones, and silicone resins), organic conditioning oils (e.g., hydrocarbon oils, polyolefins, and fatty esters) or combinations thereof, or those conditioning agents which otherwise form liquid, dispersed particles in the aqueous surfactant matrix herein.

One or more oily conditioning agents are typically present at a concentration of about 0.01 wt. % to about 10 wt. %, in one embodiment of about 0.1 wt. % to about 8 wt. %, and in another embodiment of about 0.2 wt. % to about 4 wt. %, based on the weight of the personal care composition.

b. Silicone Conditioning Agent

The oily conditioning agents of the compositions can be a water-insoluble silicone conditioning agent. The silicone conditioning agent may comprise volatile silicone, non-volatile silicone, or combinations thereof. Suitable are non-volatile silicone conditioning agents. If volatile silicones are present, it will typically be incidental to their use as a solvent or carrier for commercially available forms of non-volatile silicone materials ingredients, such as silicone gums and resins. The silicone conditioning agent particles may comprise a silicone fluid conditioning agent and may also comprise other ingredients, such as a silicone resin to improve silicone fluid deposition efficiency or enhance glossiness of the hair.

Non-limiting examples of suitable silicone conditioning agents, and optional suspending agents for the silicone, are described in U.S. Reissue Pat. No. 34,584, U.S. Pat. No. 5,104,646, and U.S. Pat. No. 5,106,609. In one embodiment the silicone conditioning agents for use in the personal care compositions have a viscosity, as measured at 25° C. of about 20 to about 2,000,000 centistokes (“csk”), in another embodiment of about 1,000 to about 1,800,000 csk, in another embodiment of about 5,000 to about 1,500,000 csk, and in yet another embodiment from about 10,000 to about 1,000,000 csk.

In an opaque composition embodiment of the present invention, the personal care composition comprises a non-volatile silicone oil having a particle size as measured in the personal care composition of about 1 μm to about 50 μm. In an embodiment of the present invention for small particle application to the hair, the personal care composition comprises a non-volatile silicone oil having a particle size as measured in the personal care composition from about 100 nm to about 1 μm. A substantially clear composition embodiment of the present invention comprises a non-volatile silicone oil having a particle size as measured in the personal care composition of less than about 100 nm.

Non-volatile silicone oils suitable for use in the personal care compositions may be selected from organo-modified silicones and fluoro-modified silicones. In one embodiment of the present invention, the non-volatile silicone oil is an organo-modified silicone which comprises an organo group selected from the group consisting of alkyl groups, alkenyl groups, hydroxyl groups, amine groups, quaternary groups, carboxyl groups, fatty acid groups, ether groups, ester groups, mercapto groups, sulfate groups, sulfonate groups, phosphate groups, propylene oxide groups, and ethylene oxide groups. In one embodiment of the present invention, the non-volatile silicone oil is dimethicone.

Background material on silicones including sections discussing silicone fluids, gums, and resins, as well as manufacture of silicones, are found in Encyclopedia of Polymer Science and Engineering, vol. 15, 2d ed., pp 204-308, John Wiley & Sons, Inc. (1989).

Silicone fluids suitable for use in the personal care compositions are disclosed in U.S. Pat. No. 2,826,551, U.S. Pat. No. 3,964,500, U.S. Pat. No. 4,364,837, British Pat. No. 849,433, and Silicon Compounds, Petrarch Systems, Inc. (1984).

c. Organic Conditioning Oils

The oily conditioning agent of the personal care compositions can further include at least one organic conditioning oil, either alone or in combination with other conditioning agents, such as the silicones described above.

d. Hydrocarbon Oils

Suitable organic conditioning oils for use as conditioning agents in the personal care compositions include, but are not limited to, hydrocarbon oils having at least about 10 carbon atoms, such as cyclic hydrocarbons, straight chain aliphatic hydrocarbons (saturated or unsaturated), and branched chain aliphatic hydrocarbons (saturated or unsaturated), including polymers and mixtures thereof. Suitable straight chain hydrocarbon oils are from about C₁₂ to about C₁₉. Branched chain hydrocarbon oils, including hydrocarbon polymers, typically will contain more than 19 carbon atoms.

Specific nonlimiting examples of these hydrocarbon oils include paraffin oil, mineral oil, saturated and unsaturated dodecane, saturated and unsaturated tridecane, saturated and unsaturated tetradecane, saturated and unsaturated pentadecane, saturated and unsaturated hexadecane, polybutene, polydecene, and mixtures thereof. Branched-chain isomers of these compounds, as well as of higher chain length hydrocarbons, can also be used, examples of which include 2,2,4,4,6,6,8,8-dimethyl-10-methylundecane and 2,2,4,4,6,6-dimethyl-8-methylnonane, available from Permethyl Corporation. A suitable hydrocarbon polymer is polybutene, such as the copolymer of isobutylene and butene, which is commercially available as L-14 polybutene from Amoco Chemical Corporation.

e. Polyolefins

Organic conditioning oils for use in the personal care compositions can also include liquid polyolefins, in one embodiment liquid poly-α-olefins, in another embodiment hydrogenated liquid poly-α-olefins. Polyolefins for use herein are prepared by polymerization of C₄ to about C₁₄ olefenic monomers, in one embodiment from about C₆ to about C₁₂.

Non-limiting examples of olefenic monomers for use in preparing the polyolefin liquids herein include ethylene, propylene, 1-butene, 1-pentene, 1-hexene, 1-octene, 1-decene, 1-dodecene, 1-tetradecene, branched chain isomers such as 4-methyl-1-pentene, and mixtures thereof. Also suitable for preparing the polyolefin liquids are olefin-containing refinery feedstocks or effluents.

f. Fatty Esters

Other suitable organic conditioning oils for use as the conditioning agent in the personal care compositions include fatty esters having at least 10 carbon atoms. These fatty esters include esters with hydrocarbyl chains derived from fatty acids or alcohols. The hydrocarbyl radicals of the fatty esters hereof may include or have covalently bonded thereto other compatible functionalities, such as amides and alkoxy moieties (e.g., ethoxy or ether linkages, etc.).

Suitable examples of fatty esters include, but are not limited to, isopropyl isostearate, hexyl laurate, isohexyl laurate, isohexyl palmitate, isopropyl palmitate, decyl oleate, isodecyl oleate, hexadecyl stearate, decyl stearate, isopropyl isostearate, dihexyldecyl adipate, lauryl lactate, myristyl lactate, cetyl lactate, oleyl stearate, oleyl oleate, oleyl myristate, lauryl acetate, cetyl propionate, and oleyl adipate. Other fatty esters suitable for use in the compositions of the present invention are those known as polyhydric alcohol esters. Such polyhydric alcohol esters include alkylene glycol esters.

Still other fatty esters suitable for use in the personal care compositions are glycerides, including, but not limited to, mono-, di-, and tri-glycerides, in one embodiment di- and tri-glycerides, in another embodiment triglycerides. A variety of these types of materials can be obtained from vegetable and animal fats and oils, such as castor oil, safflower oil, cottonseed oil, corn oil, olive oil, cod liver oil, almond oil, avocado oil, palm oil, sesame oil, lanolin and soybean oil. Synthetic oils include, but are not limited to, triolein and tristearin glyceryl dilaurate.

g. Fluorinated Conditioning Compounds

Fluorinated compounds suitable for delivering conditioning to hair or skin as organic conditioning oils include perfluoropolyethers, perfluorinated olefins, fluorine based specialty polymers that may be in a fluid or elastomer form similar to the silicone fluids previously described, and perfluorinated dimethicones. Specific non-limiting examples of suitable fluorinated compounds include the Fomblin product line from Ausimont which includes HC/04, HC/25, HC01, HC/02, HC/03; Dioctyldodecyl Fluoroeptyl Citrate, commonly called Biosil Basics Fluoro Gerbet 3.5 supplied by Biosil Technologies; and Biosil Basics Fluorosil LF also supplied by Biosil Technologies.

h. Fatty Alcohols

Other suitable organic conditioning oils for use in the personal care compositions include, but are not limited to, fatty alcohols having at least about 10 carbon atoms, in one embodiment about 10 to about 22 carbon atoms, and in another embodiment about 12 to about 16 carbon atoms. Also suitable for use in the personal care compositions of the present inventions are alkoxylated fatty alcohols which conform to the general formula:

CH₃(CH₂)ₙCH₂(OCH₂CH₂)ₚOH

wherein n is a positive integer having a value from about 8 to about 20, in one embodiment about 10 to about 14, and p is a positive integer having a value from about 1 to about 30, and in one embodiment from about 2 to about 23.

i. Alkyl Glucosides and Alkyl Glucoside Derivatives

Suitable organic conditioning oils for use in the personal care compositions include, but are not limited to, alkyl glucosides and alkyl glucoside derivatives. Specific non-limiting examples of suitable alkyl glucosides and alkyl glucoside derivatives include Glucam E-10, Glucam E-20, Glucam P-10, and Glucquat 125 commercially available from Amerchol.

j. Quaternary Ammonium Compounds

Suitable quaternary ammonium compounds for use as conditioning agents in the personal care compositions include, but are not limited to, hydrophilic quaternary ammonium compounds with a long chain substituent having a carbonyl moiety, like an amide moiety, or a phosphate ester moiety or a similar hydrophilic moiety.

Examples of useful hydrophilic quaternary ammonium compounds include, but are not limited to, compounds designated in the CTFA Cosmetic Dictionary as ricinoleamidopropyl trimonium chloride, ricinoleamido trimonium ethylsulfate, hydroxy stearamidopropyl trimoniummethylsulfate and hydroxy stearamidopropyl trimonium chloride, or combinations thereof.

Examples of other useful quaternary ammonium surfactants include, but are not limited to, Quaternium-33, Quaternium-43, isostearamidopropyl ethyldimonium ethosulfate, Quaternium-22 and Quaternium-26, or combinations thereof, as designated in the CTFA Dictionary.

Other hydrophilic quaternary ammonium compounds useful in a composition of the present invention include, but are not limited to, Quaternium-16, Quaternium-27, Quaternium-30, Quaternium-52, Quaternium-53, Quaternium-56, Quaternium-60, Quaternium-61, Quaternium-62, Quaternium-63, Quaternium-71, and combinations thereof.

k. Polyethylene Glycols

Additional compounds useful herein as conditioning agents include polyethylene glycols and polypropylene glycols having a molecular weight of up to about 2,000,000 such as those with CTFA names PEG-200, PEG-400, PEG-600, PEG-1000, PEG-2M, PEG-7M, PEG-14M, PEG-45M and mixtures thereof.

Glycerin may also be used as conditioning agent in the personal care compositions. In one embodiment of the present invention, glycerin may be present in a range of about 0.01 wt. % to about 10 wt. %, based on the total weight of the personal care product. In a further embodiment of the present invention, glycerin may be present in a range of about 0.1 wt. % to about 5 wt. %, based on the total weight of the personal care product. In yet a further embodiment of the present invention, glycerin may be present in a range of about 2 wt. % to about 4 wt. %, based on the total weight of the personal care product.

### 2. Additional Components

In some embodiments, the composition of the invention is combined with a personal care composition that includes one or more components known for use in hair care or personal care products, provided that the components are physically and chemically compatible with the essential components described herein, or do not otherwise unduly impair product stability, aesthetics or performance to form a personal care product. Individual concentrations of such additional components may range from about 0.001 wt. % to about 10 wt. %, based on the weight of the personal care product.

Non-limiting examples components that can be included in the personal care composition include: natural cationic deposition polymers, synthetic cationic deposition polymers, anti-dandruff agents, gel networks (e.g., fatty alcohol/surfactant networks), particles, suspending agents, paraffinic hydrocarbons, propellants, viscosity modifiers, dyes, non-volatile solvents, water soluble diluents, water insoluble diluents, pearlescent aids, foam boosters, additional surfactants or nonionic cosurfactants, pediculocides, pH adjusting agents, perfumes, preservatives, chelants, proteins, skin active agents, sunscreens, UV absorbers, vitamins, and mixtures thereof.

a. Cellulose or Guar Cationic Deposition Polymers

The composition of the invention can be combined with personal care compositions that include cellulose or guar cationic deposition polymers to form a personal care product. Cellulose or glactomannan cationic deposition polymers are suitable for use herein. Generally, such cellulose or guar cationic deposition polymers may be present at a concentration from about 0.05 wt. % to about 5 wt. %, based on the total weight of the personal care product. Suitable cellulose or guar cationic deposition polymers have a molecular weight of greater than about 5,000. In one embodiment, the cellulose or guar cationic deposition polymers have a molecular weight of greater than about 200,000. Additionally, such cellulose or guar deposition polymers have a charge density from about 0.15 meq/g to about 4.0 meq/g at the pH of intended use of the personal care product, which pH will generally range from about pH 3 to about pH 9, and in one embodiment between about pH 4 and about pH 8. The pH of the compositions are measured neat.

Suitable cellulose or guar cationic polymers include those which conform to the following formula:

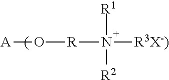

wherein A is an anhydroglucose residual group, such as a cellulose anhydroglucose residual; R is an alkylene oxyalkylene, polyoxyalkylene, or hydroxyalkylene group, or combination thereof, R¹, R², and R³ independently are alkyl, aryl, alkylaryl, arylalkyl, alkoxyalkyl, or alkoxyaryl groups, each group containing up to about 18 carbon atoms, and the total number of carbon atoms for each cationic moiety (i.e., the sum of carbon atoms in R¹, R² and R³) and in one embodiment being about 20 or less; and X is an anionic counterion. Non-limiting examples of such counterions include halides (e.g., chlorine, fluorine, bromine, iodine), sulfate and methylsulfate. The degree of cationic substitution in these polysaccharide polymers is typically from about 0.01 to about 1 cationic groups per anhydroglucose unit.

In one embodiment of the invention, the cellulose or guar cationic polymers are salts of hydroxyethyl cellulose reacted with trimethyl ammonium substituted epoxide, referred to in the industry (CTFA) as Polyquaternium 10 and available from Amerchol Corp. (Edison, N.J., USA).

Other suitable cationic deposition polymers include cationic guar gum derivatives, such as guar hydroxypropyltrimonium chloride, specific examples of which include the Jaguar series (including Jaguar C-17®) commercially available from Rhone-Poulenc Incorporated, and further including Jaguar C-500, commercially available from Rhodia.

b. Synthetic Cationic Deposition Polymers

The composition of the invention can be combined with personal care compositions that include synthetic cationic deposition polymers to form a personal care product. Generally, such synthetic cationic deposition polymers may be present at a concentration from about 0.025 wt. % to about 5 wt. %, based on the total weight of the personal care product. Such synthetic cationic deposition polymers have a molecular weight from about 1,000 to about 5,000,000. Additionally, such synthetic cationic deposition polymers have a charge density from about 0.1 meq/g to about 5.0 meq/g.

Suitable synthetic cationic deposition polymers include those which are water-soluble or dispersible, cationic, non-crosslinked, conditioning copolymers comprising: (i) one or more cationic monomer units; and (ii) one or more nonionic monomer units or monomer units bearing a terminal negative charge; wherein said copolymer has a net positive charge, a cationic charge density of from about 0.5 meq/g to about 10 meg/g, and an average molecular weight from about 1,000 to about 5,000,000.

Non-limiting examples of suitable synthetic cationic deposition polymers are described in United States Patent Application Publication US 2003/0223951 A1 to Geary et al.

c. Anti-Dandruff Actives

The composition of the invention can be combined with personal care compositions that include an anti-dandruff agent to form a personal care product. Suitable, nonlimiting examples of anti-dandruff actives include: pyridinethione salts, zinc carbonate, azoles, such as ketoconazole, econazole, and elubiol, selenium sulfide, particulate sulfur, salicylic acid and mixtures thereof. A typical anti-dandruff particulate is a pyridinethione salt. Such anti-dandruff particulate should be physically and chemically compatible with the components of the composition, and should not otherwise unduly impair product stability, aesthetics or performance.

Pyridinethione anti-microbial and anti-dandruff agents are described, for example, in U.S. Pat. No. 2,809,971; U.S. Pat. No. 3,236,733; U.S. Pat. No. 3,753,196; U.S. Pat. No. 3,761,418; U.S. Pat. No. 4,345,080; U.S. Pat. No. 4,323,683; U.S. Pat. No. 4,379,753; and U.S. Pat. No. 4,470,982.

Azole anti-microbials include imidazoles such as climbazole and ketoconazole.

Selenium sulfide compounds are described, for example, in U.S. Pat. No. 2,694,668; U.S. Pat. No. 3,152,046; U.S. Pat. No. 4,089,945; and U.S. Pat. No. 4,885,107.

Sulfur may also be used as a particulate anti-microbial/anti-dandruff agent in the anti-microbial compositions of the present invention.

The present invention may further comprise one or more keratolytic agents such as Salicylic Acid.

Additional anti-microbial actives of the present invention may include extracts of melaleuca (tea tree) and charcoal.

When present in the composition, the anti-dandruff active is included in an amount of about 0.01 wt. % to about 5 wt. %, and in one embodiment from about 0.1 wt. % to about 3 wt. %, and in another embodiment from about 0.3 wt. % to about 2 wt. %, based on the weight of the personal care product.

d. Particles

In some embodiments, the composition of the invention can be combined with a personal care composition that includes particles to form a personal care product. Particles useful in the present invention are dispersed water-insoluble particles. Particles useful in the present invention can be inorganic, synthetic, or semi-synthetic. In one embodiment the particles are incorporated at no more than about 20 wt. %, in another embodiment at no more than about 10 wt. % and in yet another embodiment at no more than 2 wt. %, by weight of the personal care product, of particles. In one embodiment the particles have an average mean particle size of less than about 300 μm.

Non-limiting examples of inorganic particles include colloidal silicas, fumed silicas, precipitated silicas, silica gels, magnesium silicate, glass particles, talcs, micas, sericites, and various natural and synthetic clays including bentonites, hectorites, and montmorillonites.

Examples of synthetic particles include silicone resins, poly(meth)acrylates, polyethylene, polyester, polypropylene, polystyrene, polyurethane, polyamide (e.g., Nylon®), epoxy resins, urea resins, acrylic powders, and the like.

Non-limiting examples of hybrid particles include sericite & crosslinked polystyrene hybrid powder, and mica and silica hybrid powder.

e. Opacifying Agents

In some embodiments, the composition of the invention can be combined with a personal care composition that includes one or more opacifying agents. Opacifying agents are typically used in cleansing compositions to impart desired aesthetic benefits to the composition, such as color. In one embodiment the opacifying agents are incorporated at no more than about 20 wt. %, in another embodiment at no more than about 10 wt. % and in yet another embodiment at no more than 2 wt. %, based on the weight of the personal care product.

Suitable opacifying agents include, for example, fumed silica, polymethylmethacrylate, micronized Teflon®, boron nitride, barium sulfate, acrylate polymers, aluminum silicate, aluminum starch octenylsuccinate, calcium silicate, cellulose, chalk, corn starch, diatomaceous earth, Fuller's earth, glyceryl starch, hydrated silica, magnesium carbonate, magnesium hydroxide, magnesium oxide, magnesium trisilicate, maltodextrin, microcrystalline cellulose, rice starch, silica, titanium dioxide, zinc laurate, zinc myristate, zinc neodecanoate, zinc rosinate, zinc stearate, polyethylene, alumina, attapulgite, calcium carbonate, calcium silicate, dextran, nylon, silica silylate, silk powder, soy flour, tin oxide, titanium hydroxide, trimagnesium phosphate, walnut shell powder, or mixtures thereof. The above mentioned powders may be surface treated with lecithin, amino acids, mineral oil, silicone oil, or various other agents either alone or in combination, which coat the powder surface and render the particles hydrophobic in nature.

The opacifying agents may also comprise various organic and inorganic pigments. The organic pigments are generally various aromatic types including azo, indigoid, triphenylmethane, anthraquinone, and xanthine dyes. Inorganic pigments include iron oxides, ultramarine and chromium or chromium hydroxide colors, and mixtures thereof.

f. Suspending Agents

In some embodiments, the composition of the invention can be combined with a personal care composition that includes a suspending agent (i.e. stabilizer) at concentrations effective for suspending water-insoluble material in dispersed form in the compositions or for modifying the viscosity of the composition, to form a personal care product. Such concentrations generally range from about 0.1 wt. % to about 10 wt. %, and in one embodiment from about 0.3 wt. % to about 5.0 wt. %, based on the total weight of the personal care product, of suspending agent.

Suspending agents useful herein include anionic polymers and nonionic polymers. Useful herein are vinyl polymers such as cross linked acrylic acid polymers with the CTFA name Carbomer.

Other optional suspending agents include crystalline suspending agents which can be categorized as acyl derivatives, long chain amine oxides, and mixtures thereof. These suspending agents are described in U.S. Pat. No. 4,741,855. These suspending agents include ethylene glycol esters of fatty acids having from about 16 to about 22 carbon atoms. Also suitable are the ethylene glycol stearates, both mono and distearate, but particularly the distearate containing less than about 7% of the mono stearate.

Other suitable suspending agents include alkanol amides of fatty acids, in one embodiment having from about 16 to about 22 carbon atoms, in another embodiment having about 16 to 18 carbon atoms, suitable for use herein include, but are not limited to, stearic monoethanolamide, stearic diethanolamide, stearic monoisopropanolamide and stearic monoethanolamide stearate.

Other long chain acyl derivatives include long chain esters of long chain fatty acids (e.g., stearyl stearate, cetyl palmitate, etc.); long chain esters of long chain alkanol amides (e.g., stearamide diethanolamide distearate, stearamide monoethanolamide stearate); and glyceryl esters (e.g., glyceryl distearate, trihydroxystearin, tribehenin) a commercial example of which is Thixin R available from Rheox, Inc. Long chain acyl derivatives, ethylene glycol esters of long chain carboxylic acids, long chain amine oxides, and alkanol amides of long chain carboxylic acids in addition to the materials listed above may be used as suspending agents.

g. Paraffinic Hydrocarbons

In some embodiments, the composition of the invention can be combined with a personal care composition that includes one or more paraffinic hydrocarbons to form a personal care product. Paraffinic hydrocarbons suitable for use in compositions of the present invention include those materials which are known for use in hair care or other personal care compositions, such as those having a vapor pressure at 1 atm of equal to or greater than about 21° C. (about 70° F.). Non-limiting examples include pentane and isopentane.

h. Propellants

In some embodiments, the composition of the invention can be combined with a personal care composition that includes one or more propellants to form a personal care product. Propellants suitable for use in compositions of the present invention include those materials which are known for use in hair care or other personal care compositions, such as liquefied gas propellants and compressed gas propellants. Suitable propellants have a vapor pressure at 1 atm of less than about 21° C. (about 70° F.). Non-limiting examples of suitable propellants are alkanes, isoalkanes, haloalkanes, dimethyl ether, nitrogen, nitrous oxide, carbon dioxide, and mixtures thereof.

i. Other Optional Components

In some embodiments, the composition of the invention can be combined with a personal care composition that includes one or more fragrances to form a personal care product. The fragrances are used for aesthetic and can be present in an amount of about 0.25 wt. % to about 2.5 wt. %, based on the total weight of the personal care product.

In some nonlimiting embodiments, the composition of the invention can be combined with a personal care composition that includes water-soluble and/or water-insoluble vitamins such as vitamins B1, B2, B6, B12, C, pantothenic acid, pantothenyl ethyl ether, panthenol, biotin and their derivatives, and vitamins A, D, E, and their derivatives, to form a personal care product. The compositions of the present invention may also contain water-soluble and water-insoluble amino acids such as asparagine, alanine, indole, glutamic acid and their salts, and tyrosine, tryptamine, lysine, histadine and their salts.

In some embodiments the composition of the invention can be combined with a personal care composition that includes a mono- or divalent salt such as sodium chloride, to form a personal care product.

In some embodiments the composition of the invention can be combined with a personal care composition that includes chelating agents, e.g., EDTA, to form a personal care product. The chelating agent functions to potentiate preservatives and is present in an active amount of up to about 0.5 wt. %, based on the total weight of the personal care product.

In some embodiments the composition of the invention can be combined with a personal care composition that includes materials useful for hair loss prevention and hair growth stimulants or agents, to form a personal care product.

In some embodiments the composition of the invention can be combined with a personal care composition that includes one or more of viscosity modifiers, dyes, non-volatile solvents, water soluble diluents, gel networks (e.g., fatty alcohol/surfactant networks), water insoluble diluents, pearlescent aids, foam boosters, additional surfactants or nonionic cosurfactants, pediculocides, pH adjusting agents, preservatives, proteins, skin active agents, sunscreens, UV absorbers, or mixtures thereof, to form a personal care product.

### Exemplary Embodiments

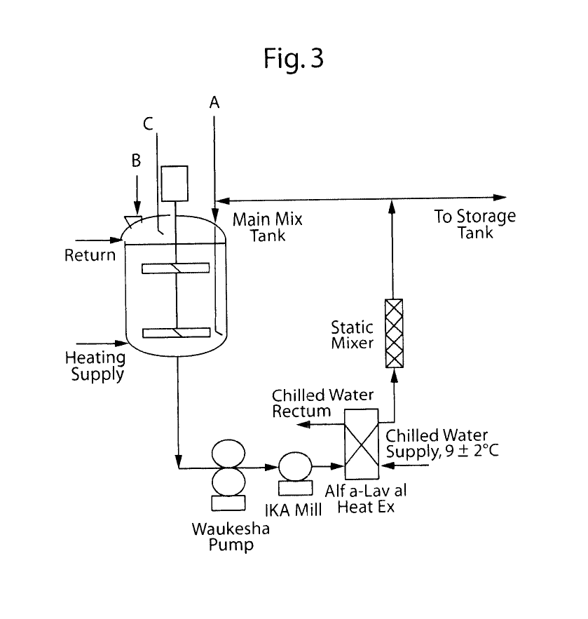

In an exemplary embodiment, the compositions of the invention are produced using the system depicted in FIG. 3. In this embodiment, detersive surfactant (e.g., SLS, SLE1S, SLE3S, or mixtures thereof) and water are introduced to the mix tank through dip tube A, and agitated at a rate sufficient to ensure good homogenization without causing aeration (e.g., 100 rpm in a 130 L double impeller baffled tank). Preservatives (e.g. at least about 0.25 wt. % sodium benzoate, or about 5 ppm to about 15 ppm of methylchloroisothiazolinone (KATHON®), or mixtures thereof, based on the total weight of the composition) and acid (e.g., about 0.5 wt. % to about 1.5 wt. % of 6N HCl, based on the total weight of the composition) are added to the mix tank through dip tube B and agitated (e.g., 40 rpm to 50 rpm in a 130 L double impeller baffled tank). The contents of the mix tank are heated to about 54° C. to about 86° C., in one embodiment from about 60° C. to about 70° C., using the heating jacket, and CMEA crystals are added to the mix tank through dip tube B. Alternatively, neutralized, preserved surfactant is heated before the addition of the CMEA. Optionally, the CMEA is preheated before being added to the mix tank. The rate of agitation is doubled during the addition of CMEA and then maintained until the CMEA is dispersed. The composition in the mix tank is then cooled to about 22° C. to about 85° C., and in one embodiment from about 27° C. to about 40° C., using the cooling jacket, and, The cooled composition is then pumped out of the mix tank to a storage tank. Alternatively, an in-line heat exchanger can function as the cooling device while the composition is being pumped to the storage tank.

### Examples

The compositions illustrated in the following Examples illustrate specific embodiments of the compositions of the present invention, but are not intended to be limiting thereof. Other modifications can be undertaken by the skilled artisan without departing from the spirit and scope of this invention.

The compositions illustrated in the following Examples are prepared by conventional formulation and mixing methods, examples of which are described above All exemplified amounts are listed as weight percents and exclude minor materials such as diluents, preservatives, color solutions, imagery ingredients, botanicals, and so forth, unless otherwise specified.

The following examples are representative of suitable dispersion compositions of the invention.

| | | EXAMPLE | | | | | | | | | |
|---|---|---|---|---|---|---|---|---|---|---|---|
| Formula | | 1 | 2 | 3 | 4 | 5 | 6 | 7 | 8 | 9 | 10 |
| Sodium Lauryl Sulfate | A | 0.0 | 0.0 | 0.0 | 14.5 | 0.0 | 0.0 | 15.0 | 0.0 | 0.0 | 8.3 |
| Sodium Laureth (1) Sulfate | B | 9.9 | 0.0 | 9.8 | 0.0 | 0.0 | 0.0 | 0.0 | 9.1 | 0.0 | 0.0 |
| Sodium Laureth (3) Sulfate | C | 12.2 | 23.1 | 8.2 | 0.0 | 8.8 | 11.4 | 0.0 | 0.0 | 14.0 | 10.7 |
| Ammonium Cocoyl | D | 1.0 | 0.0 | 0.0 | 0.0 | 11.0 | 13.3 | 0.0 | 4.5 | 0.0 | 0.0 |
| Isethionate | | | | | | | | | | | |
| Cocamide MEA (CMEA) | E | 6.80 | 10.55 | 9.57 | 8.59 | 12.75 | 8.31 | 12.75 | 6.80 | 6.80 | 9.11 |
| Sodium Benzoate | F | 0.25 | 0.25 | 0.25 | 0.25 | 0.25 | 0.25 | 0.25 | 0.25 | 0.25 | 0.25 |
| Methylchloroisothiazolinone/ | G | 0.033 | 0.033 | 0.033 | 0.033 | 0.033 | 0.033 | 0.033 | 0.033 | 0.033 | 0.033 |
| Methylisothiazolinone | | | | | | | | | | | |
| Hydrochloric Acid | H | as | as | as | as | as | as | as | as | as | as |
| | | needed | needed | needed | needed | needed | needed | needed | needed | needed | needed |
| Water | | QS to | QS to | QS to | QS to | QS to | QS to | QS to | QS to | QS to | QS to 100% |
| | | 100% | 100% | 100% | 100% | 100% | 100% | 100% | 100% | 100% | |

A Sodium Lauryl Sulfate available from Cognis Corp. as Standapol WAQ-LC, 29 wt. % active
B Sodium Laureth (1) Sulfate available from Cognis Corp. as Standapol ES-1, 25 wt. % active
C Sodium Laureth (3) Sulfate available from Cognis Corp. as Standapol ES-3, 28 wt. % active
D Ammonium Cocoyl Isethionate available from BASF Chemicals as Jordapon ACI 30G, 30 wt. % active
E Cocamide MEA available from Cognis Corp. as Comperlan CMEA, 85 wt. % active
F Sodium Benzoate available from DSM Special Products as Sodium Benzoate, 100% active
G Methylchloroisothiazolinone/Methylisothiazolinone available from Rohm & Haas as Kathon CG, 1.5 wt. % active (Listed as added wt. %, not as active wt. %)
H Hydrochloric Acid 6N available from Mallinckrodt Baker Inc. as Hydrochloric Acid 6N Solution (Listed as added wt. %, not as active wt. %)

| | | EXAMPLE | | | | | | | | | |
|---|---|---|---|---|---|---|---|---|---|---|---|
| Formula | | 11 | 12 | 13 | 14 | 15 | 16 | 17 | 18 | 19 | 20 |
| Sodium Lauryl Sulfate | A | 0.0 | 11.6 | 0.0 | 7.1 | 0.0 | 9.9 | 0.0 | 0.0 | 11.8 | 7.9 |
| Sodium Laureth (1) Sulfate | B | 14.3 | 0.0 | 0.0 | 0.0 | 8.6 | 0.0 | 9.6 | 22.1 | 0.0 | 5.6 |
| Sodium Laureth (3) Sulfate | C | 0.0 | 11.2 | 0.0 | 8.0 | 0.0 | 14.3 | 0.0 | 0.0 | 0.0 | 5.1 |
| Ammonium Cocoyl | D | 7.4 | 0.0 | 24.0 | 9.0 | 10.1 | 0.0 | 14.4 | 0.0 | 8.0 | 0.0 |
| Isethionate | | | | | | | | | | | |
| Cocamide MEA (CMEA) | E | 12.75 | 12.75 | 12.75 | 9.95 | 9.64 | 6.80 | 8.41 | 7.65 | 6.80 | 9.47 |
| Sodium Benzoate | F | 0.25 | 0.25 | 0.25 | 0.25 | 0.25 | 0.25 | 0.25 | 0.25 | 0.25 | 0.25 |
| Methylchloroisothiazolinone/ | G | 0.033 | 0.033 | 0.033 | 0.033 | 0.033 | 0.033 | 0.033 | 0.033 | 0.033 | 0.033 |
| Methylisothiazolinone | | | | | | | | | | | |
| Hydrochloric Acid | H | as | as | as | as | as | as | as | as | as | as |
| | | needed | needed | needed | needed | needed | needed | needed | needed | needed | needed |
| Water | | QS to | QS to | QS to | QS to | QS to | QS to | QS to | QS to | QS to | QS to 100% |
| | | 100% | 100% | 100% | 100% | 100% | 100% | 100% | 100% | 100% | |

A Sodium Lauryl Sulfate available from Cognis Corp. as Standapol WAQ-LC, 29 wt. % active
B Sodium Laureth (1) Sulfate available from Cognis Corp. as Standapol ES-1, 25 wt. % active
C Sodium Laureth (3) Sulfate available from Cognis Corp. as Standapol ES-3, 28 wt. % active
D Ammonium Cocoyl Isethionate available from BASF Chemicals as Jordapon ACI 30G, 30 wt. % active
E Cocamide MEA available from Cognis Corp. as Comperlan CMEA, 85 wt. % active
F Sodium Benzoate available from DSM Special Products as Sodium Benzoate, 100% active
G Methylchloroisothiazolinone/Methylisothiazolinone available from Rohm & Haas as Kathon CG, 1.5 wt. % active (Listed as added wt. %, not as active wt. %)
H Hydrochloric Acid 6N available from Mallinckrodt Baker Inc. as Hydrochloric Acid 6N Solution (Listed as added wt. %, not as active wt. %)

| | | EXAMPLE | | | | | | | | | |
|---|---|---|---|---|---|---|---|---|---|---|---|
| Formula | | 21 | 22 | 23 | 24 | 25 | 26 | 27 | 28 | 29 | 30 |
| Sodium Lauryl Sulfate | A | 8.3 | 0.0 | 24.7 | 12.0 | 11.5 | 0.0 | 12.7 | 0.0 | 0.0 | 6.3 |
| Sodium Laureth (1) Sulfate | B | 0.9 | 13.0 | 0.0 | 0.0 | 10.4 | 0.0 | 10.1 | 10.8 | 0.0 | 11.9 |
| Sodium Laureth (3) Sulfate | C | 0.0 | 0.0 | 0.0 | 0.0 | 0.0 | 0.0 | 0.0 | 10.8 | 0.0 | 0.0 |
| Ammonium Cocoyl | D | 9.9 | 0.0 | 0.0 | 12.9 | 1.6 | 15.0 | 0.0 | 0.0 | 21.4 | 0.0 |
| Isethionate | | | | | | | | | | | |
| Cocamide MEA (CMEA) | E | 12.75 | 12.75 | 6.80 | 8.94 | 6.80 | 10.37 | 10.63 | 12.75 | 6.80 | 6.80 |
| Sodium Benzoate | F | 0.25 | 0.25 | 0.25 | 0.25 | 0.25 | 0.25 | 0.25 | 0.25 | 0.25 | 0.25 |
| Methylchloroisothiazolinone/ | G | 0.033 | 0.033 | 0.033 | 0.033 | 0.033 | 0.033 | 0.033 | 0.033 | 0.033 | 0.033 |
| Methylisothiazolinone | | | | | | | | | | | |
| Hydrochloric Acid | H | as | as | as | as | as | as | as | as | as | as |
| | | needed | needed | needed | needed | needed | needed | needed | needed | needed | needed |
| Water | | QS to | QS to | QS to | QS to | QS to | QS to | QS to | QS to | QS to | QS to 100% |
| | | 100% | 100% | 100% | 100% | 100% | 100% | 100% | 100% | 100% | |

A Sodium Lauryl Sulfate available from Cognis Corp. as Standapol WAQ-LC, 29 wt. % active
B Sodium Laureth (1) Sulfate available from Cognis Corp. as Standapol ES-1, 25 wt. % active
C Sodium Laureth (3) Sulfate available from Cognis Corp. as Standapol ES-3, 28 wt. % active
D Ammonium Cocoyl Isethionate available from BASF Chemicals as Jordapon ACI 30G, 30 wt. % active
E Cocamide MEA available from Cognis Corp. as Comperlan CMEA, 85 wt. % active
F Sodium Benzoate available from DSM Special Products as Sodium Benzoate, 100% active
G Methylchloroisothiazolinone/Methylisothiazolinone available from Rohm & Haas as Kathon CG, 1.5 wt. % active (Listed as added wt. %, not as active wt. %)
H Hydrochloric Acid 6N available from Mallinckrodt Baker Inc. as Hydrochloric Acid 6N Solution (Listed as added wt. %, not as active wt. %)

EXAMPLE

| Formula | | 31 | 32 |
|---|---|---|---|
| Sodium Lauryl Sulfate | A | 8.3 | 0.0 |
| Sodium Laureth (1) Sulfate | B | 0.9 | 13.0 |
| Sodium Laureth (3) Sulfate | C | 0.0 | 0.0 |
| Ammonium Cocoyl | D | 9.9 | 0.0 |
| Isethionate | | | |
| Cocamide MEA (CMEA) | E | 12.75 | 12.75 |
| Sodium Benzoate | F | 0.25 | 0.25 |
| Methylchloroisothiazolinone/ | G | 0.033 | 0.033 |
| Methylisothiazolinone | | | |
| Hydrochloric Acid | H | as | as |
| | | needed | needed |
| Water | | QS to | QS to |
| | | 100% | 100% |

A Sodium Lauryl Sulfate available from Cognis Corp. as Standapol WAQ-LC, 29 wt. % active
B Sodium Laureth (1) Sulfate available from Cognis Corp. as Standapol ES-1, 25 wt. % active
C Sodium Laureth (3) Sulfate available from Cognis Corp. as Standapol ES-3, 28 wt. % active
D Ammonium Cocoyl Isethionate available from BASF Chemicals as Jordapon ACI 30G, 30 wt. % active
E Cocamide MEA available from Cognis Corp. as Comperlan CMEA, 85 wt. % active
F Sodium Benzoate available from DSM Special Products as Sodium Benzoate, 100% active
G Methylchloroisothiazolinone/Methylisothiazolinone available from Rohm & Haas as Kathon CG, 1.5 wt. % active (Listed as added wt. %, not as active wt. %)
H Hydrochloric Acid 6N available from Mallinckrodt Baker Inc. as Hydrochloric Acid 6N Solution (Listed as added wt. %, not as active wt. %)

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A composition comprising liquid high activity micellar or lamellar phase liquid crystals of cocamide monoethanolamine (CMEA), a detersive surfactant, and water.

2. The composition of claim 1, wherein the composition comprises a molar ratio range of CMEA to detersive surfactant of about 1:1 to about 1:20, and a molar ratio of CMEA to water of about 1:50 to about 1:1000.

3. The composition of claim 1, wherein the composition comprises a molar ratio range of CMEA to detersive surfactant of about 1:1 to about 1:5, and a molar ratio of CMEA to water of about 1:50 to about 1:250.

4. The composition of claim 1, wherein the composition comprises about 5 wt. % to about 25 wt. % of CMEA, about 10 wt. % to about 30 wt. % of detersive surfactant, and about 55 wt. % to about 85 wt % of water, based on the total weight of the composition.

5. The composition of claim 1, wherein the detersive surfactant comprises at least one anionic surfactant, wherein the anionic surfactant has an ethoxylate level of about 0 to about 10 and an anion level of about 1 to about 6.

6. The composition of claim 5, wherein the detersive surfactant comprises an anion selected from the group consisting of sulfates, sulfonates, sulfosuccinates, isethionates, carboxylates, phosphates, phosphonates, and mixtures thereof.

7. The composition of claim 6, wherein the detersive surfactant comprises sodium lauryl sulfate.

8. The composition of claim 7, wherein the composition comprises about 6.8 wt. % to about 12.5 wt. % of CMEA, about 12 wt. % to about 22 wt. % of sodium lauryl sulfate, and about 62.5 wt. % to about 81.2 wt % of water, based on the total weight of the composition.

9. The composition of claim 6, wherein the detersive surfactant comprises sodium laureth sulfate 1-ethoxylate.

10. The composition of claim 9, wherein the composition comprises about 6.8 wt. % to about 12.5 wt. % of CMEA, about 12 wt. % to about 22 wt. % of sodium laureth sulfate-1 ethoxylate, and about 62.5 wt. % to about 81.2 wt % of water, based on the total weight of the composition.

11. The composition of claim 6, wherein the detersive surfactant comprises sodium laureth sulfate 3-ethoxylate.

12. The composition of claim 11, wherein the composition comprises about 6.8 wt. % to about 12.5 wt. % of CMEA, about 12 wt. % to about 22 wt. % of sodium laureth sulfate 3-ethoxylate, and about 62.5 wt. % to about 81.2 wt % of water, based on the total weight of the composition.

13. The composition of claim 6, wherein the detersive surfactant further comprises a hydrotrope in an amount of about 0.5 wt. % to about 5.0 wt. %, based on the total weight of the composition.

14. The composition of claim 6, wherein the detersive surfactant further comprises a compound selected from the group consisting of amphoteric surfactants, zwitterionic surfactants, cationic surfactants, nonionic surfactants, and mixtures thereof.

15. A composition comprising about 5 wt. % to about 25 wt. % of cocamide monoethanolamine (CMEA), about 10 wt. % to about 30 wt. % of a detersive surfactant, and about 55 wt. % to about 85 wt. % of water, wherein the composition is substantially free of hexagonal phase liquid crystals.

16. A method comprising:
(a) adding cocamide monoethanolamine (CMEA) to a solution of a neutralized, preserved detersive surfactant at a temperature of about 54° C. to about 86° C. to form a composition comprising high activity micellar or lamellar phase liquid crystals; and,
(b) optionally cooling the composition;
wherein the composition has a molar ratio of CMEA to detersive surfactant of about 1:1 to about 1:20 and a molar ratio of CMEA to water of about 1:50 to about 1:1000.

17. The method of claim 16, further comprising adding the cooled composition to a personal care composition comprising a component selected from the group consisting of conditioning agents, natural cationic deposition polymers, synthetic cationic deposition polymers, anti-dandruff agents, gel networks, particles, suspending agents, paraffinic hydrocarbons, propellants, viscosity modifiers, dyes, non-volatile solvents, water soluble diluents, water insoluble diluents, pearlescent aids, foam boosters, surfactants, pediculocides, pH adjusting agents, perfumes, preservatives, chelants, proteins, skin active agents, sunscreens, UV absorbers, vitamins, and mixtures thereof to form a personal care product.

18. The method of claim 17, wherein CMEA is present in the personal care product in an amount of about 0.025 wt. % to about 5 wt. %, based on the total weight of the personal care product.

19. A method of making a personal care product comprising: combining,
(a) a personal care composition comprising a component selected from the group consisting of conditioning agents, natural cationic deposition polymers, synthetic cationic deposition polymers, anti-dandruff agents, gel networks, particles, suspending agents, paraffinic hydrocarbons, propellants, viscosity modifiers, dyes, non-volatile solvents, water soluble diluents, water insoluble diluents, pearlescent aids, foam boosters, surfactants, pediculocides, pH adjusting agents, perfumes, preservatives, chelants, proteins, skin active agents, sunscreens, UV absorbers, vitamins, and mixtures thereof; with,
(b) a composition comprising high activity micellar or lamellar phase liquid crystals of cocamide monoethanolamine (CMEA), a detersive surfactant, and water.

20. The method of claim 19, wherein the composition comprising lamellar phase liquid crystals is present in an amount of up to about 30 wt. %, based on the total weight of the product.
